(19) United States
(12) Patent Application Publication
(10) Pub. No.: US 2023/0418046 A1
(43) Pub. Date: Dec. 28, 2023

(54) HEADLIGHT MAGNIFIER WITH TWO MAGNIFICATIONS

(71) Applicant: Gi Chun KIM, Gwangmyeong-si (KR)

(72) Inventor: Gi Chun KIM, Gwangmyeong-si (KR)

(21) Appl. No.: 18/037,062

(22) PCT Filed: May 24, 2022
(86) PCT No.: PCT/KR2022/007333

§ 371 Date: May 15, 2023

(30) Foreign Application Priority Data
Jun. 15, 2021 (KR) 10-2021-0077721

(51) Int. Cl. G02B 25/02; F21V 21/084

(52) U.S. Cl. CPC G02B 25/02; F21V 21/084

(57) ABSTRACT

The present invention relates to a headlight magnifier which is worn on an operator's head during a precise manual operation, and allows the operator to magnify and see a work area while illuminating the work area, such that the operator may use the present invention to see the work area in a comfortable position at two magnifications with the help of excellent lighting.

### TECHNICAL FIELD

The present invention relates to a headlight magnifier which is worn on an operator's head during a precise manual operation, and allows the operator to magnify and see a work area while illuminating the work area, and, for example, relates to a headlight magnifier for enabling doctors to magnify affected sites while illuminating the affected sites when the doctors examine patients or operate.

### BACKGROUND ART

The following two conditions are important upon performing precise manual work. First, proper lightings are required to illuminate a work area, and Second, magnifiers are required to magnify the work area. A lighting worn on an operator's head among the lightings is called a headlight. In general, there are many headlight products having a magnifier capable of magnifying the work area.

### DISCLOSURE

### Technical Problem

The headlight magnifier in the related art has low effectiveness since the magnifier can be magnified only at a single magnification. In some cases, there is a headlight magnifier viewed at two magnifications by combining two magnifiers. However, the angle formed by optical axes of eye-lenses of the two magnifiers is excessively great, thereby almost departing from the human field of view, and accordingly, the operator fails to view the two magnifiers by moving only the pupils.

### Technical Solution

In order to solve the above problem, according to the present invention, upon combining a light source with a magnifier having two different magnifications, the first magnifier of the two magnifiers is set as a magnifier in which the optical axis of the eye-lens intersects with the optical axis of the objective lens, and the optical axis of the eye-lens of the first magnifier is positioned near a horizontal plane passing through a center of the eyeball. The second magnifier is set as a magnifier in which the optical axis of the eye-lens coincides with the optical axis of the objective lens, and the optical axis of the eye-lens of the second magnifier is arranged to be at a comfortable angle within the wearer's field of view. In addition, the light source is positioned on a sagittal suture plane of the operator and rotatably arranged on the sagittal suture plane and the angle of the light source is adjusted, so as to illuminate all fields of view through the magnifier with two magnifications.

### Advantageous Effects

According to the headlight magnifier, the wearer can view both of the magnifiers with the two magnifications at a comfortable angle. In addition, the light source illuminates both of the fields of view of the magnifiers, so that the wearer can view the work area with the two magnifications under sufficiently bright illuminance.

### DESCRIPTION OF DRAWINGS

FIG. 1 is a perspective view showing a state in which a headlight equipped with two magnifiers having different magnifications proposed by the present invention is worn on the head.

FIG. 2 is an enlarged perspective view of a portion in which the magnifiers are equipped according to the headlight magnifier equipped with the two magnifiers having the different magnifications.

FIG. 3 is a side view showing only the optical elements of a first magnifier of the two magnifiers having the different magnifications.

FIG. 4 is a sectional view cut in a sagittal plane to view an internal structure of the headlight magnifier equipped with the two magnifiers having the different magnifications.

FIG. 5 is a side view showing a state in which a wearer wears the headlight equipped with the two magnifiers having the different magnifications and views a work area through a first magnifier.

FIG. 6 is a side view showing a state in which the wearer wears the headlight equipped with the two magnifiers having the different magnifications and views the work area through a second magnifier.

### BEST MODE

The present invention, as the best mode for embodiments, provides a headlight magnifier including: a light source configured to generate light and includes a connection unit hinged with a headband around a rotational axis perpendicular to a sagittal suture plane, in which the connection unit is formed on an outer circumferential surface thereof with three locking steps forming two valleys; a headband hinged with the connection unit of the light source around the rotational axis, coupled to a first magnifier in a vicinity of the hinged portion, and worn on a head of a wearer; a stopper supported by the headband and including a protrusion coming into contact with the outer circumferential surface of the connection unit of the light source; a first magnifier coupled to the headband and having an objective lens with an optical axis and an eye-lens with an optical axis in which the optical axes intersect with each other at an angle of 48 degrees, and the optical axis of the eye-lens passes through a center of an eyeball of the wearer; and a second magnifier coupled to a lower portion of the first magnifier, and having an objective lens with an optical axis and an eye-lens with an optical axis in which the optical axes coincide with each other, and the optical axis passes through the center of the eyeball of the wearer, wherein a point at which the light source illuminates when the light source rotates one pitch between the valleys of the locking steps of the connection unit moves by an arc length, that is, a separation distance obtained by multiplying a working distance by an angle for rotating the head to view a magnifier having a different magnification.

### MODE FOR INVENTION

Hereinafter, the embodiments according to the present invention will be described with reference to the accompanying drawings.

First, the light source will be described with reference to FIGS. 1, 2, 4, 5 and 6. The light source 1 generates light and collects the light using a reflector 12 to illuminate a work area 0. The light may be collected by using a lens instead of the reflector. The work area refers to a predetermined distance in terms of the working distance as a most comfortable for each operator which is generally arranged between 30 and 50 centimeters in the case of doctors and depends on each operator. A light source connection unit 11 protrudes from an upper part of the light source, so as to be hinged with a headband 2 formed in a flat cylindrical shape and having a rotational axis 112 perpendicular to the sagittal suture plane. Three locking steps 111 protrude from ab outer circumferential surface of the light source connection unit 11, and a first valley 113 and a second valley 114 are formed between the three locking steps. The light source connecting unit 11 slides in contact with a protrusion 221 of a stopper 22. When the light source 1 rotates around the rotational axis 112, and when the protrusion 221 of the stopper 22 is positioned in the two valleys between the three locking steps 111 protruding from the light source connection unit, the rotation may stop stably, and accordingly the rotation is interrupted twice intermittently. Specifically, in FIG. 4, the protrusion 221 of the stopper comes into contact with the second valley 114. At this time, the light source 1 may illuminate the field of view of a second magnifier corresponding to the working distance. When the light source 1 rotates clockwise and the protrusion 221 of the stopper comes into contact with the first valley 113, the light source may illuminate the work area 0 at the working distance to which an objective lens 34 of the first magnifier 3 is directed.

The headband 2 refers to a part worn on the operator's head, and is a ‘C’-shaped headband hinged to the light source connection unit 11 and extending to the occipital region via the forehead and the top of the head. In the present invention, the ‘C’-shaped headband is taken as an example, but any type of headband may be used to be worn on the head. However, it is an important factor for the present invention in that the rotational movement may be temporarily stopped when the light source 1 rotates on the sagittal suture plane since the light source connection unit 11 and the headband are hinged around the rotational axis 112 perpendicular to the sagittal suture plane, and the stopper 22 is disposed between the light source connection unit and the headband. The stopper 22 refers to an elastic component elongating up and down, flat back and forth and having a predetermined width left and right, and is supported at rear sides of upper and lower ends in contact with the headband. As an idealized aspect, the stopper may be regarded as a simple beam shape having support points at the upper and lower ends and receiving a concentrated load at a central portion in contact with the light source connecting unit 11. When the light source connection unit 11 rotates, the stopper 22 may sag while receiving a concentrated load by one of the locking steps 111, and accordingly the locking step may pass over the protrusion 221. In other words, the protrusion 221 may stably stop only at the first valley and the second valley of the locking steps 111.

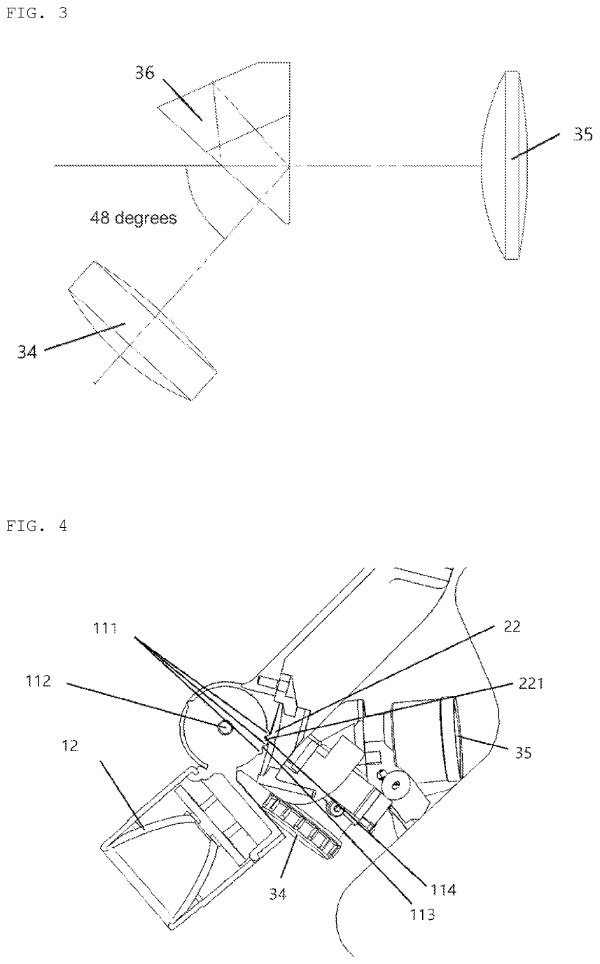

Hereinafter, the first magnifier 3 will be described with reference to FIGS. 3, 4 and 5. The first magnifier is composed of a pair of Kepler-type telescopes on left and right sides and a first magnifier frame 37 for supporting the pair of Kepler-type telescopes. The Kepler-type telescope has an erection prism 36 added between the objective lens 34 and the eye-lens 35, in which the optical axis 32 of the objective lens and the optical axis 33 of the eye-lens form an angle of 48 degrees. The first magnifier frame 37 is coupled to the headband 2 near and below the hinge portion of the headband. The optical axis 33 of the eye-lens 35 of the first magnifier passes through a center of the operator's eyeball 51 and is horizontal as a whole, and the optical axis 32 of the objective lens 34 passes through the work area 0. When the operator wearing the headlight magnifier looks at the eye-lens 35 of the first magnifier, the work area 0 at the angle of almost 48 degrees downward to which the objective lens 34 is directed may be viewed. More accurately, since the point where the optical axis 32 of the objective lens intersects with the optical axis 33 of the eye-lens is positioned in front of the operator's eyes 5, the actual work area 0 is positioned in a working distance at an angle slightly smaller than 48 degrees about the center of the operator's eyeball based on the operator's eyes 5, and accordingly, the work area 0 may be viewed. Since the protrusion 221 of the stopper comes into contact with the first valley 113, the light source 1 is directed to the work area 0 on the optical axis 32 of the objective lens of the first magnifier, so that the operator may view the work area with bright illuminance.

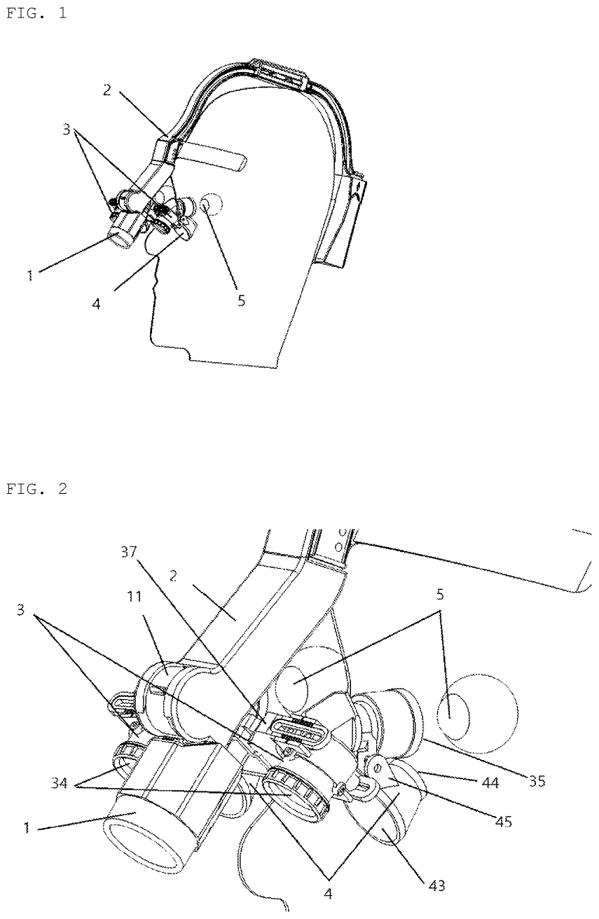

Hereinafter, the second magnifier 4 will be described with reference to FIGS. 2, 4 and 5. The optical axes 42 of the objective lens 43 and the eye-lens 44 of the second magnifier are arranged on a straight line. The optical axis 42 of the eye-lens of the second magnifier passes through the center of the eyeball 51 of the operator. The second magnifier 4 is coupled to the first magnifier 3 at a vicinity of the erect prism 36 of the first magnifier through a second magnifier support 45. In the present embodiment, the second magnifier support 45 is taken as an example to be coupled to the first magnifier, but other embodiments are also possible. In other words, the second magnifier support 45 may be directly coupled to the headband 2. The important feature is that the optical axis 42 of the eye-lens 44 of the second magnifier passes through the center of the wearer's eyeball 51, and the optical axis of the second magnifier is arranged at an angle for allowing the wearer's eyes to comfortably look down and positioned in a place for preventing the first magnifier from interfering with the field of view. In addition, the present embodiment is taken as an example in which the second magnifier has a form of a telescope having the objective lens and the eye-lens, but in some cases, the present embodiment may be provided with a magnifier or reducing glass with a single lens. The present embodiment may be provided with a telescope in which the optical axes of the objective lens and the eye-lens are almost coincident with each other by using two prisms in a Kepler-type telescope. These cases are allowed when the optical axis of the eye-lens of the second magnifier passes through the vicinity of the center of the eyeball. Returning to the present embodiment again, it is assumed that the angle formed by the optical axis 33 of the eye-lens 35 of the first magnifier 3 and the optical axis 42 of the eye-lens 44 of the second magnifier 4 is called ‘A’, and the angle formed by the optical axis 42 of the second magnifier 4 and the line extending to the work area 0 from the center of the eyeball 51 is called ‘B’. When the operator rotates the eyeball downward by the angle ‘A’, and the operator simultaneously rotates the head by the angle ‘B’, the operator may view the work area 0 through the second magnifier. However, in this case, since the light source 1 also rotates by the angle ‘B’ rotated by the head, the point illuminated by the light source may pass over the work area 0 and illuminate a point spaced away by a separation distance d, and accordingly, the field of view that is viewed by the operator through the second magnifier becomes dark. In order to solve this, when the light source 1 rotates clockwise about the rotational axis 112 by the angle corresponding to the separation distance d, the light source may illuminate the work area 0 in the field of view of the second magnifier. The above angle may allow the light source 1 to coincide with one pitch of the locking step 111 of the connection unit 11 about the rotational axis 112, that is, the angle between the first valley 113 and the second valley 114.

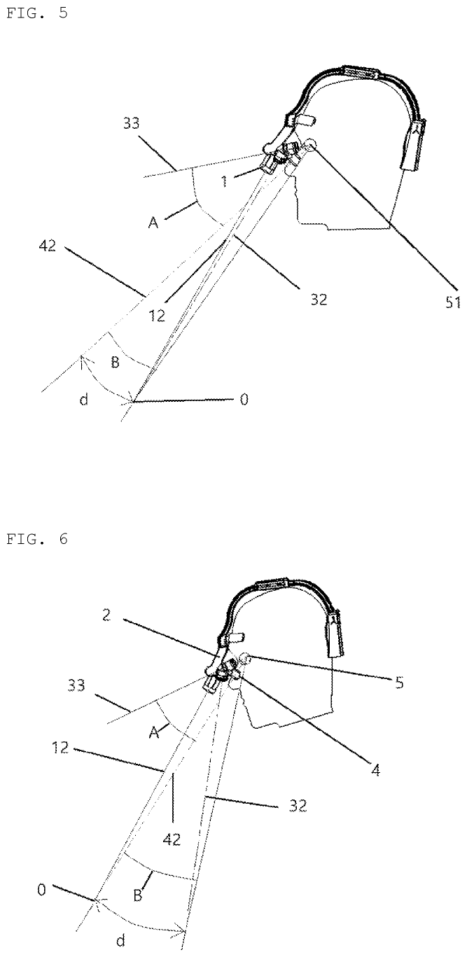

Hereinafter, the operation of the headlight magnifier proposed by the present invention will be described. Referring to FIG. 5, first, the operator may wear the headlight magnifier proposed in the present invention and view the work area 0 through the eye-lens 35 of the first magnifier 3. At this time, the light source 1 is directed to the work area 0. When the operator wants to view the work area through the second magnifier, the eyeball 5 may rotate counterclockwise by the angle ‘A’ based on a center of the eyeball 51 to allow the pupil to face the eye-lens of the second magnifier, and the head may rotate counterclockwise by the angle ‘B’, so that the work area 0 comes into the field of view of the second magnifier. When the light source 1 rotates clockwise by one pitch of the locking step 111 of the connection unit 11 about the rotational axis 112, the protrusion 221 of the stopper may move from the first valley 113 to the second valley 114 and the light source may illuminate the work area 0, so that the work area 0 may be viewed with bright illuminance. When the operator wants to view the work area 0 again through the first magnifier, the eyeball 5 may rotate clockwise by the angle ‘A’ about the center of the eyeball 51, the operator's head may rotate clockwise by the angle ‘B’, and the light source 1 may rotate counterclockwise by the one pitch of the locking step 111 about the rotational axis 112. The specification of Korean Unexamined Patent Application No. 10-2020-0165653 disclosed in Dec. 1, 2020 exemplifies an embodiment in which the angle formed by the optical axis 33 of the eye-lens of the first magnifier 3 and the optical axis 42 of the eye-lens of the second magnifier 4, that is, the angle ‘A’ is made larger by the angle ‘B’ so that the operator may view the work area through the second magnifier by rotating only the eyeball without rotating the head. However, in this invention, the eyes feel very uncomfortable since the second magnifier is excessively directed downward. The present invention improves the above inconvenience by disposing the second magnifier at an angle for allowing the eyeball to view comfortably, so that one headlight magnifying system allows comfortable view with two magnifications.

A: The angle formed by the optical axis of the eye-lens of the first magnifier and the optical axis of the eye-lens of the second magnifier

B: The point where the working distance coincides with the optical axis 42 of the second magnifier 4 and the optical axis 32 toward which the objective lens 34 of the first magnifier is directed from the center of the eyeball 51, that is, the angle formed by the line connecting to the work area 0 in FIG. 5

Separation distance d: The distance in which the light source rotates as the head rotates by the angle B and accordingly the point to which the light source is directed moves on the working distance. Arc length d=working distance×angle B

Working distance: The distance to the point the operator wants to view from the center of the operator's eyeball. The present embodiment is illustrated, as an example, such that the distance from the center of the operator's eyeball to the point where the light source axis 12 intersects with the first magnifier optical axis 32 is matched with the working distance.

It will be apparent to those skilled in the art that the present invention is not limited to the described embodiments and accompanying drawings, and various replacements, deformations and modifications are available without departing from the spirit or scope of the invention as defined in the appended claims.

1. A headlight magnifier comprising:
a light source configured to generate light and including a connection unit hinged with a headband around a rotational axis perpendicular to a sagittal suture plane, in which the connection unit is formed on an outer circumferential surface thereof with three locking steps forming two valleys;
a headband hinged with the connection unit of the light source around the rotational axis, coupled to a first magnifier in a vicinity of the hinged portion, and worn on a head of a wearer;
a stopper supported by the headband and including a protrusion coming into contact with the outer circumferential surface of the connection unit of the light source;
a first magnifier coupled to the headband, having an objective lens with an optical axis and an eye-lens with an optical axis in which the optical axes intersect with each other at an angle of 48 degrees, and the optical axis of the eye-lens passes through a center of an eyeball of the wearer; and
a second magnifier coupled to a lower portion of the first magnifier and having an objective lens with an optical axis and an eye-lens with an optical axis in which the optical axes coincide with each other, and the optical axis passes through the center of the eyeball of the wearer, wherein
a point at which the light source illuminates when the light source rotates one pitch between the valleys of the locking steps of the connection unit moves by an arc length, that is, a separation distance obtained by multiplying a working distance by an angle for rotating the head to view a magnifier having a different magnification.

2. A headlight magnifier comprising:
a light source configured to generate light and including a connection unit hinged with a headband around a rotational axis perpendicular to a sagittal suture plane, in which the connection unit is formed on an outer circumferential surface thereof with three locking steps forming two valleys;
a headband hinged with the connection unit of the light source around the rotational axis, coupled to a first magnifier in a vicinity of the hinged portion, and worn on a head of a wearer;
a stopper supported by the headband and including a protrusion coming into contact with the outer circumferential surface of the connection unit of the light source;
a first magnifier coupled to the headband, having an objective lens with an optical axis and an eye-lens with an optical axis in which the optical axes intersect with each other at an angle of 48 degrees, and the optical axis of the eye-lens passes through a center of an eyeball of the wearer; and
a second magnifier coupled to the headband and having an objective lens with an optical axis and an eye-lens with an optical axis in which the optical axes coincide with each other, and the optical axis passes through the center of the eyeball of the wearer, wherein
a point at which the light source illuminates when the light source rotates one pitch between the valleys of the locking steps of the connection unit moves by an arc length, that is, a separation distance obtained by multiplying a working distance by an angle for rotating the head to view a magnifier having a different magnification.

3. The headlight magnifier of claim 1, wherein the second magnifier includes a single lens.

4. The headlight magnifier of claim 2, wherein the second magnifier includes a single lens.
